(19) United States
(12) Patent Application Publication
(10) Pub. No.: US 2019/0117074 A1
(43) Pub. Date: Apr. 25, 2019

(54) METHODS AND SYSTEMS FOR IDENTIFYING FUNCTIONAL AREAS OF CEREBRAL CORTEX USING OPTICAL COHERENCE TOMOGRAPHY

(71) Applicants: Sean Jy-Shyang CHEN, Toronto (CA); Siu Wai Jacky MAK, Toronto (CA); SYNAPTIVE MEDICAL (BARBADOS) INC., Bridgetown (BB)

(72) Inventors: Sean Jy-Shyang CHEN, Toronto (CA); Siu Wai Jacky MAK, Toronto (CA)

(21) Appl. No.: 15/551,920

(22) PCT Filed: Nov. 2, 2016
(86) PCT No.: PCT/CA2016/051269

§ 371 Date: Aug. 18, 2017

(51) Int. Cl. A61B 5/00; A61B 34/10; A61B 34/20; G06T 7/00

(52) U.S. Cl. CPC A61B 5/0066; A61B 34/10; A61B 34/20; A61B 2576/026; A61B 5/0042; G06T 2207/30016; G06T 2207/10101; G06T 7/0014

(57) ABSTRACT

A method and system to identify function in areas of a cerebral cortex using optical coherence tomography to scan an area and compare the scan to a cytoarchitectural database of classified images. A matched image has an associated likely function. Using a navigation system, the location of the cerebral cortex scanned is determined and is associated with the likely function corresponding to the matched image. A registration module may generate an image, possibly including pre-operative scan data, of the cerebral cortex with likely functions indicated for the scanned locations.

### FIELD

The present application generally relates to scanning of a cerebral cortex using optical coherence tomography (OCT) and, in particular, using OCT to determine and identify likely function of areas of the cortex.

### BACKGROUND

In the field of medicine, imaging and image guidance are a significant component of clinical care. From diagnosis and monitoring of disease, to planning of the surgical approach, to guidance during procedures and follow-up after the procedure is complete, imaging and image guidance provides effective and multifaceted treatment approaches, for a variety of procedures, including surgery and radiation therapy. Targeted stem cell delivery, adaptive chemotherapy regimens, and radiation therapy are only a few examples of procedures utilizing imaging guidance in the medical field. Optical tracking systems, used during a medical procedure, track the position of a part of the instrument that is within line-of-site of the optical tracking camera. These optical tracking systems also require a reference to the patient to know where the instrument is relative to the target (e.g., a tumour) of the medical procedure.

Pre-operative imaging data such as Magnetic Resonance Imaging (MRI), Computerized Tomography (CT) and Positron Emission Tomography (PET), is integrated into the surgical room statically through a viewing station, or dynamically through a navigation system. The navigation system registers devices to a patient, and a patient to the pre-operative scans, allowing for instruments to be viewed on a monitor in the context of the pre-operative information.

In neurosurgery, it can be helpful to be aware of the functional areas of the cerebral cortex so as to ensure that areas associated with critical functions are avoided when planning or executing the surgical operation. Active stimulation is sometimes used to attempt to identify functional areas, but this requires keeping the patient awake during surgery. Functional MRI is sometimes used to try to identify functional areas, but this technique is subject to delay, noise, low spatial resolution, and unreliability. Functional MRI is commonly performed before the operation. Intra-op MRI also limits the type of tools that could be used in the operating room to prevent hazards due to the magnetic field from the MRI system.

### BRIEF SUMMARY

The present application describes a method for identifying and displaying anatomical functional areas of a cerebral cortex. The method includes obtaining cross-sectional cerebral cortex image data from an optical coherence tomography (OCT) scanner; comparing the cross-section cerebral cortex image data with cytoarchitectural image data from a cytoarchitectural image database to identify a match to an associated likely function; determining, based on input from a navigation system, a location on the cerebral cortex from which the cerebral cortex image data was obtained; associating the likely anatomical function with the location; generating an image of the cerebral cortex having the likely anatomical function indicated on the image at the location; and displaying the image on a display.

In another aspect, the present application describes a system to identify and display anatomical functional areas of a cerebral cortex. The system includes an optical coherence tomography (OCT) scanner to obtain cross-sectional cerebral cortex image data; a cytoarchitectural image database containing a plurality of classified images of cortical scans, each classified image being associated with a respective function; an OCT analyzer to compare the cross-sectional cerebral cortex image data with cytoarchitectural image data from the cytoarchitectural database to identify a match to a likely function; a navigation system to determine a location on the cerebral cortex from which the cerebral cortex image data was obtained; a registration module to associate the likely anatomical function with the location and to generate an image of the cerebral cortex having the likely anatomical function indicated on the image at the location; and a display to display the image.

In yet a further aspect, the present application describes non-transitory computer-readable media storing computer-executable program instructions which, when executed, configured a processor to perform the described methods.

Other aspects and features of the present application will be understood by those of ordinary skill in the art from a review of the following description of examples in conjunction with the accompanying figures.

In the present application, the term “and/or” is intended to cover all possible combination and sub-combinations of the listed elements, including any one of the listed elements alone, any sub-combination, or all of the elements, and without necessarily excluding additional elements.

In the present application, the phrase “at least one of . . . or . . . ” is intended to cover any one or more of the listed elements, including any one of the listed elements alone, any sub-combination, or all of the elements, without necessarily excluding any additional elements, and without necessarily requiring all of the elements.

### BRIEF DESCRIPTION OF THE DRAWINGS

Reference will now be made, by way of example, to the accompanying drawings which show example embodiments of the present application, and in which:

FIG. 1 shows a diagram and a cross-section OCT scan of cerebral cortical tissue;

FIG. 2 shows, in block diagram form, one example of a system for identifying functional areas of a cerebral cortex; and

FIG. 3 shows, in flowchart form, one example of a method for identifying function areas of a cerebral cortex.

Similar reference numerals may have been used in different figures to denote similar components.

### DESCRIPTION OF EXAMPLE EMBODIMENTS

In the field of medicine, imaging and image guidance are a significant component of clinical care. From diagnosis and monitoring of disease, to planning of the surgical approach, to guidance during procedures and follow-up after the procedure is complete, imaging and image guidance provides effective and multifaceted treatment approaches, for a variety of procedures, including surgery and radiation therapy. Targeted stem cell delivery, adaptive chemotherapy regimens, and radiation therapy are only a few examples of procedures utilizing imaging guidance in the medical field. Optical tracking systems, used during a medical procedure, track the position of a part of the instrument that is within line-of-site of the optical tracking camera.

Advanced imaging modalities such as Magnetic Resonance Imaging (“MRI”) have led to improved rates and accuracy of detection, diagnosis and staging in several fields of medicine including neurology, where imaging of diseases such as brain cancer, stroke, Intra-Cerebral Hemorrhage (“ICH”), and neurodegenerative diseases, such as Parkinson's and Alzheimer's, are performed. As an imaging modality, MRI enables three-dimensional visualization of tissue with high contrast in soft tissue without the use of ionizing radiation. This modality is often used in conjunction with other modalities such as Ultrasound (“US”), Positron Emission Tomography (“PET”) and Computed X-ray Tomography (“CT”), by examining the same tissue using the different physical principles available with each modality. CT is often used to visualize bony structures and blood vessels when used in conjunction with an intra-venous agent such as an iodinated contrast agent. MRI may also be performed using a similar contrast agent, such as an intra-venous gadolinium-based contrast agent which has pharmaco-kinetic properties that enable visualization of tumors and break-down of the blood brain barrier. These multi-modality solutions can provide varying degrees of contrast between different tissue types, tissue function, and disease states. Imaging modalities can be used in isolation, or in combination to better differentiate and diagnose disease.

In neurosurgery, for example, brain tumors are typically excised through an open craniotomy approach guided by imaging. The data collected in these solutions sometimes consists of CT scans with an associated contrast agent, such as iodinated contrast agent, as well as MRI scans with an associated contrast agent, such as gadolinium contrast agent. Also, optical imaging is often used in the form of a microscope to differentiate the boundaries of the tumor from healthy tissue, known as the peripheral zone.

When conducting a neurosurgical operation, the surgical team wants to avoid certain critical areas of the brain that are key to basic functions. For example, when planning a trajectory for accessing a tumor, the surgeon may wish to avoid traversing an area fundamental to speech, sight, motor functions, or other basic functional areas, so as to avoid potential damage to those critical areas and the possibility of post-surgery loss of function. Accordingly, the surgical team often wishes to identify the functional areas of the brain so as to avoid certain areas.

One technique for identifying functional areas is to engage in active stimulation to determine the functions of particular areas. For example, the patient may be instructed to carry out a function, such as speaking, and an electrical stimulus may be applied to areas to see if it impacts the patient's ability to perform the function. This technique necessarily involves keeping the patient conscious and alert while the cranium is opened and exposed so as to stimulate areas of the brain. This technique can prolong the surgery and introduce risks and complications.

Another technique that has been tried is the use of MRI to measure changes in blood oxygenation as a surrogate for neural activity. This sometimes terms “functional” MRI, or fMRI. This technique relies on there being a correlation between blood oxygenation and activity in the brain. That correlation is somewhat loose and comes with a lag in occurrence and detection, meaning that it is not a consistently reliable indicator of activity. The fMRI technique suffers from noise and spurious correlations, and accurate registration alignment of the functional signal with an anatomical image can be problematic.

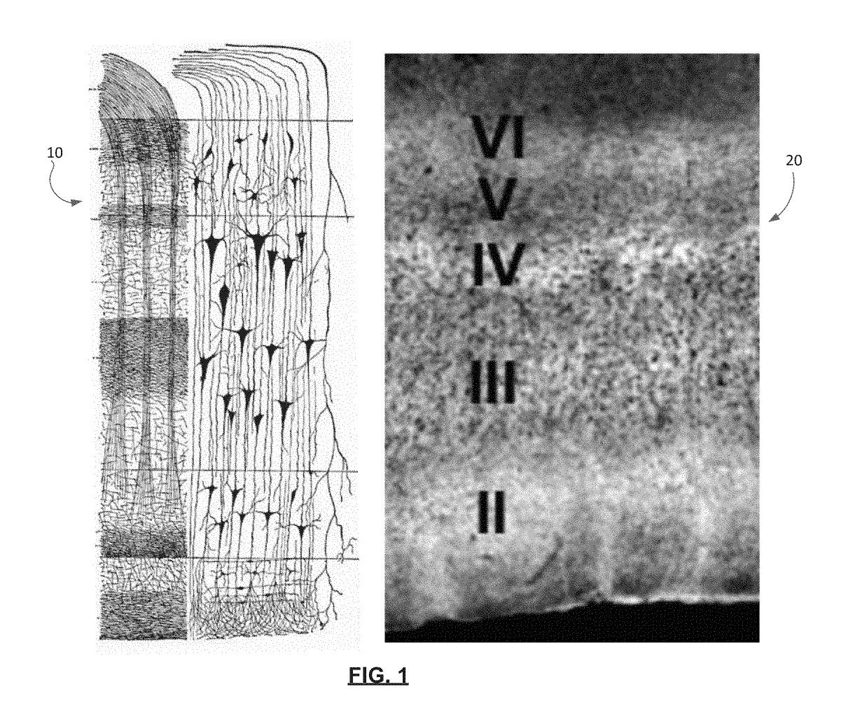

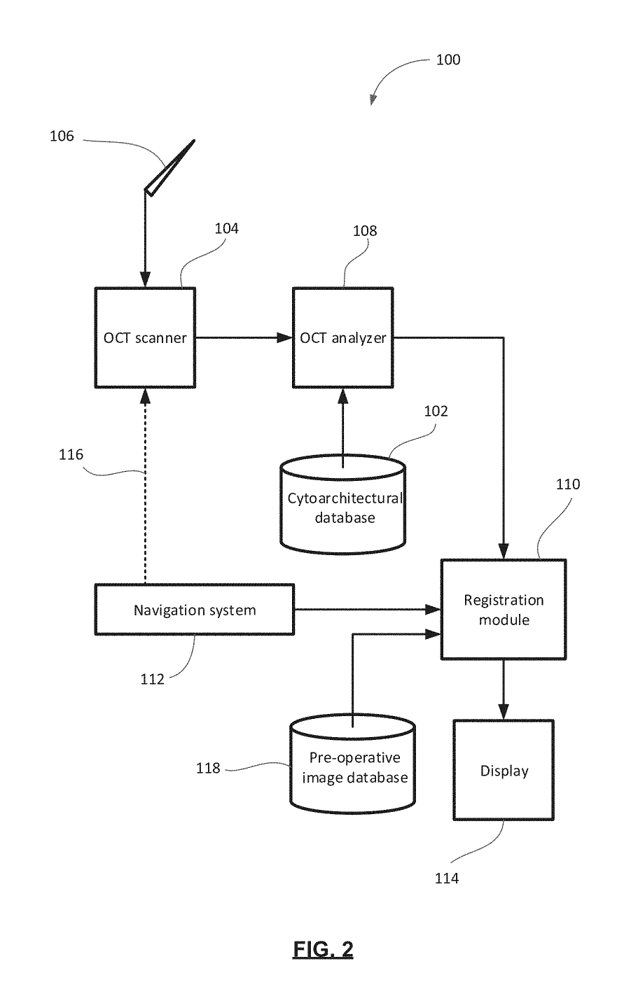

Some work has been done to correlate functions to cytoarchitecture of the cerebral regions, i.e. the layers of cortical cellular structure. FIG. 1 shows an example of the banding of cortical layers. On the left is an illustrated diagram 10 indicating the banding of the cortical layers: Henry Gray, Anatomy of the Human Body, (1918), FIG. 754. On the right is an example of a cross-sectional image 20 of a scanned cerebral cortex: C. Magnian, et al., “Cytoarchitecture of cortex imaged by Optical Coherence Tomography”, Poster FIG. 2A, Organization for Human Brain Mapping, Seattle, Wash., USA, Jun. 16-20, 2013. The image 20 was obtained using optical coherence tomography (OCT). A region's specific cytoarchitecture, or the organization of the layered cortical cellular structures, may be considered a signature that indicates the associated function of that region. To this end, cytoarchitectonic maps have been developed. Cytoarchitecture-based region differentiation is one of the most precise indicators of brain function, and is considered superior to some commonly used macroscopic landmarks indicators (e.g. sulci, gyri). Additional background on cytoarchitecture and mapping to function may be found in (1) von Economo C, Koskinas G N “Die Cytoarchitektonik der Hirnrinde des Erwachsenen Menschen: Textband and Atlas mit 112 Mikrophotographischen Tafeln.”, 1925, Springer, Vienna; (2) Amunts K, Schleicher A, Zilles K. “Cytoarchitecture of the cerebral cortex—More than localization”, Neurolmage, 2007 October, 37(4):1061-5; and (3) Bludau S, Eickhoff S B, Mohlberg H, Caspers S, Laird A R, Fox P T, et al. “Cytoarchitecture, probability maps and functions of the human frontal pole”, Neurolmage, 2014 June, 93 Pt 2:260-75, for example, the contents of which are hereby incorporated by reference.

OCT scanning may be used to image to a depth of 2-3 mm, which is sufficient to intraoperatively obtain imaging of the cerebral cortical layers. Existing cross-sectional OCT techniques can readily image at sufficient depth to include the six layers of the cerebral cortex. OCT can also identify the neuronal structures without the use of contrast agents and distinctly image the cortical layers in vivo. In some cases, a minimally-invasive OCT side firing probe may be used and inserted into the top 2-3 mm of the sample, e.g. in a sulcus between gyri, to do a higher resolution scan with even greater contrast.

Reference is now made to FIG. 2, which shows a simplified block diagram of an example system 100 for identifying functional regions of the cerebral cortex. The system 100 includes a cytoarchitecture database 102. The database 102 includes a plurality of classified cytoarchitectural images that include a link between each image, its associated function and the region on the cerebral cortex where it is found. Each function is associated with a plurality of images, and the plurality of images common to an associated function features one or more common layer characteristics and/or neuronal structures that distinguish the plurality of images associated with that function from the plurality of images associated with other functions.

The system 100 further includes an OCT scanner 104. OCT scanning in the medical field was originally focused on retinal calls. More recently, OCT scanning has been applied in other field, such as for dermatology for imaging the blood vessel networks proximate suspected skin cancer lesions. The OCT scanner 104 may include a probe 106 or scanning wand that a user manipulates to direct the scanning light beam to a desired area. In the case of a neurological scan, the OCT scanner 104 obtains and outputs a cross-sectional OCT image of the cerebral cortex showing the sub-surface cortical anatomy, such as the cortical layers and neuronal structures, to a depth of 2-3 mm.

The system 100 also includes an OCT analyzer 108 to receive the cross-sectional image(s) from the OCT scanner 104. The OCT analyzer 108, in some embodiments, finds a best-fit match between an OCT image and the images in the cytoarchitectural database 102. The OCT analyzer 108 may use image similarity comparison, such as Pearson's correlation and mutual information, to determine a best fit with one or more images in the database 102. In some implementations, the image analyzer 108 may use cytoarchitectonic probability maps in determining a likely function associated with the region in the OCT image, where the probability map shows the likelihood (in probabilistic numerical terms) that an OCT image from the OCT scanner 104 matches the cytoarchitecture of known and classified regions of the cerebral cortex having assigned likely functions

The OCT analyzer 108 and database 102 may, in one embodiment, include a set of cross-sectional OCT images, where each OCT image is tagged with the image's associated function. It may be further labelled by its region on the cerebral cortex and/or with its layer number. The OCT analyzer 108 may be configured to directly compare the cross-sectional image from the OCT scanner 104 with the stored images in the database looking for a best-fit match, with at least a threshold level of confidence, based on an image comparison metric. The process may include some image registration or resampling, and statistical determination of the most likely match(es) based on the compared metrics. The metrics may include, for example, cross-correlation, mutual information, etc.

In some embodiments, the OCT analyzer 108 and database 102 may include a trained classifier that, based on a set of training images that have been tagged and labelled, is configured to determine the likely function of an input cross-sectional image from the OCT scanner 104. In some examples, the classifier may use a nearest-neighbour analysis. In some examples, the classifier may use a random decision forest analysis. Other classification mechanisms may be used to classify the scanned OCT image and to thereby determine its associated likely function.

The system 100 further includes a navigation system 112, a registration module 110 and at least one display 114. The navigation system 112 may include an optical navigation system or other such systems for tracking the location of objects in the operating theatre in real-time. That is, the navigation system 112 is capable of determining the three-dimensional location of at least one medical device, such as the probe 106, vis-à-vis a patient reference. An optical navigation system may track the location of devices using stereoscopic cameras, a plurality of fiducials mounted to the device-to-be-tracked, and image recognition software capable of identifying the fiducials in images captured by the cameras. The optical navigation system uses an initial registration process to define a coordinate space and the location of the patient within that coordinate space. The patient may be fixed in location using a clamp or other devices for ensuring the patient maintains a constant location. A patient reference marker may be attached to the clamp or other equipment, such as a device positioner, secured in place to assist the navigation system in optically determining the location of the patient and the relative location of other devices based on fiducials patterns. The details of navigation systems and their use in tracking devices in the operating theatre will be familiar to those of ordinary skill in the art.

The image analyzer 108 may output the likely function associated with a given OCT image together with information regarding the OCT scanning operation associated with the OCT image. For example, the image analyzer 108 may receive information from the OCT scanner 104 regarding a time stamp associated with the OCT image obtained using the probe 106. That is, the OCT image is obtained at a specified point in time. The OCT scanner 104 may have been synchronized to a common time base with at least some other systems in a prior time synchronization operation. As an example, the OCT scanner 104 may receive a time sync signal 116 from the navigation system 112 to lock the OCT scanner's internal timing circuit to a common time base with other portions of the system 100. In other examples, the time sync signal 116 may be received from OCT analyzer 108 or other parts of the system 100. Irrespective of the mechanism used for time sync, the OCT scanner 104 provides the OCT analyzer 108 with the OCT image and its associated time stamp so that the time at which the OCT image was captured is preserved.

The navigation system 112 may track the location of the probe 106 relative to the patient, e.g. in a navigation coordinate space. The navigation system 112 may further track other devices.

The registration module 110 receives, from the OCT analyzer 108, at least the likely function and the time stamp associated with the OCT image with which the likely function is associated. The registration module 110 further receives navigation information from the navigation system 112. In some cases, the registration module 110 may request navigation information from the navigation system 112 based on the time stamp received from the OCT analyzer 108. That is, the registration module 110 may request that the navigation system identify the location of the probe 106 at the time indicated by the time stamp. The registration module 110 is shown separately for clarity, but it may form part of the OCT analyzer 108, the navigation system 112, or another module or device,

The registration module 110 correlates the location of the probe 106 at the time of the time stamp with the likely function determined by the OCT analyzer, so as to map the likely function to a specific location on the cerebral cortex. The registration module 110 may receive a plurality of likely functions each associated with distinct time stamps. In this manner, the registration module may build a map of likely functions associated with different areas of the cerebral cortex.

In some embodiments, the location of the probe 106 specified by the navigation system 112 identifies a region or general area of the cerebral cortex that is then also used by the OCT analyzer 108 as a factor in determining the likely function. For example, if the probe 106 is located in the frontal lobe area, then the determination of likely function may take that into account when assessing whether the scanned image data matches images in the database. In this example, the region knowledge may indicate that the match is unlikely to be related to visual function, and the OCT analyzer 108 may reduce the likelihood weighting or probability associated with that function as a result. Accordingly, the determination of likely function may take into account a best fit between the scanned image and images in the database, but that matching operation may include weighting the probabilities of a match based on the general location of the probe and the known general areas of the cerebral cortex in which particular functions are to be found.

The registration module 110 may receive data from other image sources, such as a pre-operative image database 118 containing pre-operative image data, e.g. magnetic resonance imaging (MRI) scans, computerized axial tomography (CAT) scans, etc. The registration module 110 may align the pre-operative image data with navigation system data by transforming one or more sets of data into a common three-dimensional data space. The registration module 110 may then generate one or more output two-dimensional view of the data in the three-dimensional data space for rendering on the display 114. In this manner, the surgeon and other operating room personnel may view the displayed image data during the operation procedure. In particular, the registration module 110 may visually indicate the likely functions mapped to areas of the cerebral cortex on the displayed images. This may permit the planning and execution of operative procedures so as to avoid likely critical function areas. The likely functions may be indicated by text labels in some embodiments, by colour codes in some embodiments, by shading in some embodiments, or using any other visual indicators or combination of visual indicators.

In some embodiments, the OCT analyzer 108 determines a confidence level associated with the likely function. That is, the OCT analyzer 108 may numerically indicate the degree to which the OCT image is strongly correlated with a likely function, i.e. the degree of confidence with which its image characteristics can be matched to images characteristic of the likely function using, for example, cytoarchitectonic probability maps. The OCT analyzer 108 may provide that confidence level information or probability map to the registration module 110. The registration module 110 may be configured to visually display the confidence level associated with a likely function. For example, where the likely function is indicated using a color code, the confidence level may be indicated by the intensity and/or transparency of the colour, e.g. a more transparent shading is indicative of a lower confidence level while a more solid non-transparent shading is indicative of a higher confidence level. Other techniques may be used to visually indicate the confidence level associated with a likely function, including text.

In some embodiments, a single OCT image may result in a set of one or more likely functions, each having an associated probability. The collection of two or more OCT images from nearby locations may be used to generate a map of probably functions for the area, and the relative probabilities of the two or more scans may be used to develop a refined probability map for the likely function of the area. In this manner, the system 100 may build and refine a map of likely functions for the cerebral cortex.

Further refinement to the map of likely functions, or to the probability map associated with a single scan, may be based on additional information such as the scans cortical location and brain lobe, which may impact the probabilities that the area is associated with certain functions and not with others.

In some cases, the system 100 further includes a microscope/camera trained upon the surgical area to provide a close-up view of the surgical zone. This live feed may be mapped, based on registration with the navigation system 112, to the same coordinate space as the data from the OCT analyzer 108, thereby enabling display of the live video feed of the surgical zone with likely functional areas displayed as an overlay to the video feed.

The display of the likely function information on the display 114 may take many forms in various embodiments. For example, in some cases a list of cortical functions and their associated probabilities may be displayed for each OCT scan. In some cases, a user may be prompted to select one of the displayed functions, at which point the system 100 then associates the OCT scan with that function. In some examples, the map of likely functions is dynamically displayed on a model of the cerebral cortex displayed on the display 114, and the likely functions and their relative probabilities may be dynamically updated as new OCT scans are taken and analyzed.

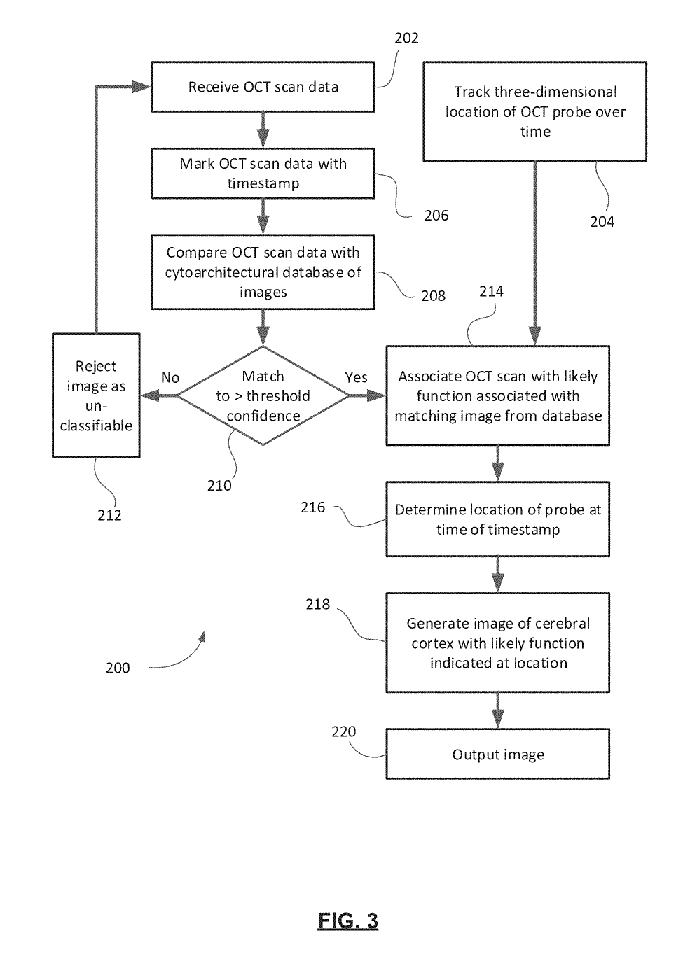

Reference is now made to FIG. 3, which shows, in flowchart form, an example process 200 for identifying functional areas of the cerebral cortex. The process 200 may be implemented by one or more computing devices suitably programmed with software and having communications subsystems for receiving and outputting data. The process 200 includes an operation 202 of receiving OCT scan data from an OCT scanner. The OCT scan data is cross-sectional image data from a cerebral cortex. The image data includes at least the cortical layers of a specific location of the cerebral cortex. A probe with a scanning end is used in the specific location to obtain the OCT scan data. The OCT scan data thus obtained is marked with a timestamp in operation 206.

While operations 204 and 206 are undertaken, in operation 204 a navigation system tracks the location of the probe over time. The location data is tracked and stored in association with timestamps indicating the time at which the location data was obtained. Thus, the navigation system determines the location of the probe and, in particular, an identifiable feature of the problem, such as a set of fiducial markers. The navigation system further includes a three-dimensional model of the probe so that the location of the tip or scanning end of the probe may be determined based on the determined location of the fiducial markers.

In operation 208, the OCT scan data is compared with the images of a cytoarchitectural database. In some cases, as described above, the comparison is carried out using a classifier that has been trained by a set of previously classified images, such that the OCT analyzer is not directly comparing the OCT scan data with individual images in the cytoarchitectural database but rather is classifying the OCT scan data based on a classifier that has been trained using the images in the cytoarchitectural database.

In operation 210, the system assesses whether it has been able to match the OCT scan data to an image or set of images from the cytoarchitectural database with sufficient confidence, i.e. whether it has been able to classify the OCT scan data by identifying at least one associated likely function with a minimum probability. In other words, it assess whether the quality of the match or classification meets a threshold confidence level. The assessment of the quality of the match may be based on any one of a number of image analysis and feature matching algorithms.

If, in operation 210, a match cannot be made with sufficient confidence, i.e. the likely function associated with the OCT scan data cannot be determined to at least the threshold degree of confidence, then in operation 212 the OCT scan data is rejected as unclassifiable. The system may output an error notification to indicate to an operator that the recently collected OCT scan data was not classifiable, such as an auditory or visual alert. The process 200 then returns to operation 202 to receive further OCT scan data. It will be understood that more than one likely function may be identified with sufficient confidence in operation 208.

If, however, in operation 210 a match is found with sufficient confidence, then in operation 214 the likely function associated with the matching cytoarchitectural image(s) is associated with the OCT scan data.

As noted above, the assessment of whether a match meets a sufficient confidence threshold of probability may also take into account the general location at which the OCT scan data was taken. For example, the likelihood of a match may be weighted based on the general area of the cerebral cortex at which the data was obtained and whether certain functions are known to be located in other areas of the cerebral cortex.

In operation 216 the location of the probe at the time at which the OCT scan data was collected, i.e. based on the timestamp, is obtained from the navigation system. The navigation system is able to determine, based on modeling of the probe and detection of its location relative to the patient reference object, from what location the OCT scan data was obtained. Thus, the system is able to associate the likely function with a specific location of the cerebral cortex. Also, as noted above, the general location of the OCT scan may influence the likelihood that the scan is indicative of certain functions based on a known correlation between areas of the cerebral cortex and certain functions.

In operation 218, an image is generated showing at least one view of the cerebral cortex. The image may include pre-operative image data, such as MRI data, CAT scan data, or other imaging data. The image includes at least a visual indicator of the likely function associated with the specific location of the cerebral cortex. The image is output in operation 220, for example to a display for viewing by personnel in the operating room.

Certain adaptations and modifications of the described embodiments can be made. Therefore, the above discussed embodiments are considered to be illustrative and not restrictive.

What is claimed is:

1. A method for identifying and displaying anatomical functional areas of a cerebral cortex, the method comprising:
obtaining cross-sectional cerebral cortex image data from an optical coherence tomography (OCT) scanner;
comparing the cross-section cerebral cortex image data with cytoarchitectural image data from a cytoarchitectural image database to identify a match to an associated likely anatomical function;
determining, based on input from a navigation system, a location on the cerebral cortex from which the cerebral cortex image data was obtained;
associating the likely anatomical function with the location;
generating an image of the cerebral cortex having the likely anatomical function indicated on the image at the location; and
displaying the image on a display.

2. The method claimed in claim 1, wherein the obtaining cross-sectional cerebral cortex image data operation further includes receiving the cross-sectional cerebral cortex image data from an OCT probe placed proximate the cerebral cortex.

3. The method claimed in claim 2, wherein the OCT probe has one or more markers trackable by the navigation system, and wherein the determining the location includes determining, by the navigation system, the three-dimensional location of the OCT probe relative to the cerebral cortex.

4. The method claimed in claim 1, further comprising assigning a time stamp to the cerebral cortex image data, and wherein determining the location comprises determining, by the navigation system, the location of an OCT probe at a time corresponding to the time stamp.

5. The method claimed in claim 1, wherein comparing includes determining that an image from the database matches the cerebral cortex image data to more than a threshold level of confidence.

6. The method claimed in claim 1, wherein the cytoarchitectural image data from a cytoarchitectural image database comprises classification data associating likely functions to image characteristics, and wherein comparing comprises using a classifier to identify the match to the associated likely function based on the classification data.

7. The method claimed in claim 1, wherein comparing further comprises weighting candidate likely anatomical functions based on the location on the cerebral cortex from which the cerebral cortex image data was obtained.

8. The method claimed in claim 1, wherein generating an image further includes incorporating pre-operative image data from a pre-operative scan.

9. The method claimed in claim 1, wherein generating includes marking the image of the cerebral cortex at the location with a colour corresponding to the likely function.

10. The method claimed in claim 1, wherein the obtaining, comparing, determining and associating are performed with respect to a plurality of locations on the cerebral cortex, and wherein generating further comprises building a function map indicating the likely function associated with each of the plurality of locations.

11. A system to identify and display anatomical functional areas of a cerebral cortex, the system comprising:
an optical coherence tomography (OCT) scanner to obtain cross-sectional cerebral cortex image data;
a cytoarchitectural image database containing a plurality of classified images of cortical scans, each classified image being associated with a respective function;
an OCT analyzer to compare the cross-sectional cerebral cortex image data with cytoarchitectural image data from the cytoarchitectural database to identify a match to a likely function;
a navigation system to determine a location on the cerebral cortex from which the cerebral cortex image data was obtained;
a registration module to associate the likely anatomical function with the location and to generate an image of the cerebral cortex having the likely anatomical function indicated on the image at the location; and
a display to display the image.

12. The system claimed in claim 11, wherein the OCT scanner includes an OCT probe to be placed proximate the cerebral cortex when obtaining the cross-sectional cerebral cortex image data.

13. The system claimed in claim 12, wherein the OCT probe has one or more markers trackable by the navigation system, and wherein the navigation system is configured to determine a three-dimensional location of the OCT probe relative to the cerebral cortex.

14. The system claimed in claim 11, wherein the OCT scanner is configure to assign a time stamp to the cerebral cortex image data, and wherein the navigation system is configured to determine the location of an OCT probe at a time corresponding to the time stamp.

15. The system claimed in claim 11, wherein the OCT analyzer is to compare by determining that at least one of the classified images matches the cerebral cortex image data to more than a threshold level of confidence.

16. The system claimed in claim 11, wherein the registration module is to generate the image by incorporating pre-operative image data from a pre-operative scan.

17. The system claimed in claim 11, wherein the registration module is to generate the image by marking the image of the cerebral cortex at the location with a colour corresponding to the likely function.

18. A non-transitory processor-readable medium storing processor-executable instructions for identifying and displaying functional areas of a cerebral cortex, wherein the processor-executable instructions, when executed by one or more processors, cause the one or more processors to:
obtain cross-sectional cerebral cortex image data from an optical coherence tomography scanner;
compare the cross-section cerebral cortex image data with cytoarchitectural image data from a cytoarchitectural image database to identify a match to an associated likely function;
determine, based on input from a navigation system, a location on the cerebral cortex from which the cerebral cortex image data was obtained;
associate the likely function with the location;
generate an image of the cerebral cortex having the likely function indicated on the image at the location; and
display the image on a display.

19. The non-transitory processor-readable medium claimed in claim 18, wherein the instructions to obtain includes instructions receive the cross-sectional cerebral cortex image data from an OCT probe placed proximate the cerebral cortex.

20. The non-transitory processor-readable medium claimed in claim 19, wherein the OCT probe has one or more markers trackable by the navigation system, and wherein the navigation system is configured to determine a three-dimensional location of the OCT probe relative to the cerebral cortex.

21. The non-transitory processor-readable medium claimed in claim 18, further comprising instructions that, when executed, cause the one or more processors to assign a time stamp to the cerebral cortex image data and determine the location by determining the location of an OCT probe at a time corresponding to the time stamp.

22. The non-transitory processor-readable medium claimed in claim 18, wherein the instructions include instructions to compare by determining that an image from the database matches the cerebral cortex image data to more than a threshold level of confidence.
